(19) United States
(12) Patent Application Publication
(10) Pub. No.: US 2014/0308261 A1
(43) Pub. Date: Oct. 16, 2014

(54) HLA-DR7 HY epitope and method for treating leukaemia

(71) Applicant: Etablissement Français du Sang, La Plaine Saint Denis Cedex (FR)

(72) Inventors: Assia El Jaafari, Vourles (FR); Dominique Rigal, Saint Didier Au Mont D'or (FR); Diane Scott, London (GB)

(21) Appl. No.: 14/359,442

(22) PCT Filed: Nov. 21, 2012
(86) PCT No.: PCT/EP2012/073275

§ 371 Date: May 20, 2014

(30) Foreign Application Priority Data
Nov. 21, 2011 (FR) 1160578

(51) Int. Cl. C07K 7/08

(52) U.S. Cl. CPC C07K 7/08
USPC 424/93.71; 530/326; 536/23.5; 514/21.5; 514/19.3; 435/320.1; 435/69.1; 435/373; 435/325

(57) ABSTRACT

This invention concerns HY epitopic polypeptides specifically presented by the HLA-DR7 molecule, a method for preparing these epitopic polypeptides, isolated T-lymphocytes capable of specifically recognizing an epitope from these polypeptides or from a polypeptide comprising the complete sequence of the protein encoded by the RPS4Y gene and presented by the HLA-DR7 molecule expressed on the surface of antigen-presenting cells, a method for preparing these T-lymphocytes, as well as the use of these epitopic polypeptides and these T-lymphocytes as medicaments, in particular for the treatment of cancers of immune cells.

This invention concerns HY epitopic polypeptides specifically presented by the HLA-DR7 molecule, a method for preparing these epitopic polypeptides, isolated T-lymphocytes capable of specifically recognizing these epitopic polypeptides, a method for preparing these T-lymphocytes, as well as the use of these epitopic polypeptides and T-lymphocytes as medicaments, especially in the treatment of cancer of the cells of the immune system.

Minor histocompatibility antigens (mHAg) are peptides presented by HLA molecules and encoded by autosomal genes or present on sex chromosomes (Simpson and Roopenian (1997) Curr. Opin. Immunol. 9:655-661). Their disparity between donor/receiver pairs otherwise presenting the same HLA typing is associated with an increased risk of transplant failure and/or graft versus host disease (GVHD) after bone marrow transplant (Hambach and Goulmy, 2005: Curr. Opin. Immunol. 17:202-210); (Falkenburg et al., 2003: Exp. Hematol. 31:743-751). The HY antigen is one of these mHAg. Encoded by genes located on the Y chromosome, it will be targeted by T-lymphocytes from female donors after a bone marrow transplant between different genders in a pair of siblings who are otherwise HLA-identical (Vogt et al., 2002: Blood 99:3027-3032).

However, this mHAg mismatching between a donor and a receiver can also have advantages. Indeed, it can induce a graft versus leukaemia (GVL) or graft versus tumour (GVT) effect, depending on the tissue distribution of these mHAg. Therefore, mHAg expression exclusively on malignant cells or haematopoietic cells can, preferentially, lead to a GVL or GVT effect, while a mHAg expressed ubiquitously would rather lead to a GVHD effect (Ferrara et al. (2009) Lancet. 373:1550-1561). For example, it has been demonstrated that the two HLA class I-restricted mHAg, HA-1 and HA-2, for which the expression is limited to haematopoietic cells, induce a GVL effect by stimulating cytotoxic CD8⁺ T-lymphocytes from the donor, which recognize the malignant cells of the receiver (Marijt et al., 2003: Proc. Natl. Acad. Sci. USA 100:2742-2747); (Hambach et al., 2006: Leukemia 20:371-374).

Another way of specifically targeting the receiver's malignant cells to obtain a GVL effect is associated with the fact that the mHAg epitope is presented by a specific type of HLA molecule. While HLA class 1 molecules are ubiquitously expressed, HLA class II molecules are mainly expressed on antigen-presenting cells, B-lymphocytes, activated T-lymphocytes and endothelial cells. Leukaemia, myeloma and other tumour cells that express HLA class II molecules can therefore be targeted by T-lymphocytes that specifically recognize a mHAg presented by an HLA class II molecule without resulting in harmful effects on normal tissues (Rutten et al., 2008: Leukemia 22:1387-1394). Therefore, it is particularly helpful to be able to identify mHAgs restricted to HLA class II molecules in light of cancer treatment by immunotherapy.

Several HLA class I-restricted mHAgs have been identified in humans by the use of different methods such as screening of plasmid cDNA libraries, elution of peptides bound to HLA, and genetic linkage analysis. Quite recently, a new strategy by whole genome association scan has reportedly provided effective characterisation of 10 new HLA class I-restricted mHAgs. However, it is more difficult to identify HLA class II-restricted mHAgs.

In humans, among the candidate genes that might encode an HY antigen, it has been demonstrated that only six, RPS4Y, UPS9Y, DDX3Y, UTY, TMSB4Y and SMCY, encoded clinically pertinent HY epitopes. However, most of them are HLA class II-restricted. Only two, DDX3Y and RPS4Y, encode HLA class II-restricted mHAgs. In addition, to date, very few peptides have been characterised.

Spierings et al. (2003: Lancet 362:610-615) describes the identification of an HY antigen recognized by T helper cells in a woman who had an HLA-identical stem cell transplantation from a male donor. This HY antigen of sequence SEQ ID NO: 3 is from the protein RPS4Y, and interacts with the HLA-DR83*0301 molecule.

Similarly, Ivanov et al. (2005: Clin. Cancer Res. 11:1694-1703) describes the identification of an HY antigen of sequence SEQ ID NO: 2, derived from the protein RPS4Y, capable of inducing a cytotoxic T-lymphocyte response that targets proliferative lymphocytes. This antigen interacts with the HLA-B*5201 molecule.

Due to significant polymorphism of HLA class II molecules, it is particularly important to identify new mHAg epitopic peptides so that more patients can be treated, especially new mHAg epitopic peptides capable of binding HLA alleles that are frequently observed in the general population. But, the peptides described in Spierings et al. (2003) and lvanov et al. (2005) have the disadvantage of interacting with HLA alleles that are rarely expressed in the general population.

An HY epitopic peptide which is restricted to the HLA class II HLA-DR7, more frequently expressed in the general population than HLA-DR83*0301 or HLA-B*5201, and which is encoded by the RPS4Y gene, was unexpectedly identified by the inventors in a male patient suffering from moderate GVHD after a bone marrow transplant from his HLA-identical sister. This peptide was identified as being recognized by a population of CD4⁺ T-lymphocytes presenting both helper and cytotoxic functions against the cells displaying the HLA-DR7-restricted peptide. This population of CD4⁺ T-lymphocytes was isolated by the inventors after several cycles of stimulation of CD4⁺/CD8⁺ double positive T-lymphocytes from skin biopsies from this transplanted patient, with antigen-presenting cells from the same patient.

Therefore, this invention concerns an isolated polypeptide comprising:

a) the TGKIINFIKFDTGNL sequence (SEQ ID NO: 1), or

b) a peptide fragment of at least 9 consecutive amino acids of the sequence SEQ ID NO: 1, or

c) a variant of the sequence SEQ ID NO: 1, or of the fragment according to b) differing from the sequence SEQ ID NO: 1, or from the fragment according to b) by conservative substitution of one, two or three amino acids; or

d) a peptidomimetic of the sequence SEQ ID NO: 1, of the fragment according to b) or of the variant according to c),

said peptide fragment, said peptidomimetic and said variant having the capacity to activate T-lymphocytes when it is presented by the HLA-DR7 molecule;

said polypeptide not comprising the complete sequence of the protein encoded by the RPS4Y gene.

The present invention also concerns an isolated polypeptide comprising:

a) the TGKIINFIKFDTGNL sequence (SEQ ID NO: 1), or

b) a peptide fragment of at least 9 consecutive amino acids of the sequence SEQ ID NO: 1, or

c) a variant of the sequence SEQ ID NO: 1, or of the fragment according to b) differing from the sequence SEQ ID NO: 1, or from the fragment according to b) by conservative substitution of one, two or three amino acids; or

d) a peptidomimetic of the sequence SEQ ID NO: 1, of the fragment according to b) or of the variant according to c),

said peptide fragment, said peptidomimetic and said variant having the capacity to activate T-lymphocytes when it is presented by the HLA-DR7 molecule;

said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3).

It also concerns a nucleic acid comprising or consisting of a sequence encoding this polypeptide, as well as a vector comprising this nucleic acid operatively bound to one or more elements enabling the expression of the polypeptide.

Another object of the present application is related to a method for producing the polypeptide according to the invention, comprising the steps consisting of:

a) chemically synthesizing the said polypeptide or inducing the expression of the said polypeptide by a cell comprising the nucleic acid or the vector according to the invention; and

b) retrieving the said polypeptide obtained in step a).

In addition, this invention concerns an isolated T-lymphocyte capable of specifically recognizing an epitope from the polypeptide of the invention or a polypeptide comprising the complete sequence of the protein encoded by the RPS4Y gene and presented by the HLA-DR7 molecule expressed on the surface of antigen-presenting cells, as well as a method of in vitro preparation of such a T-lymphocyte, comprising the steps of:
T-lymphocytes co-culture with antigen-presenting cells that express on their surface the HLA-DR7 molecule, to which is attached an epitope from the polypeptide of the invention or a polypeptide comprising the complete sequence of the protein encoded by the RPS4Y gene; and
retrieval of T-lymphocytes from the said co-culture.

It also concerns a pharmaceutical composition comprising (i) the polypeptide of the invention or a T-lymphocyte of the invention and (ii) a pharmaceutically acceptable vehicle, as well as a medicament comprising the polypeptide of the invention or a T-lymphocyte of the invention.

This invention also concerns the isolated polypeptide of the invention or a polypeptide comprising the complete sequence of the protein encoded by the RPS4Y gene, or the isolate T-lymphocyte of the invention, for its use in the treatment of cancer of the cells of the immune system.

### DETAILED DESCRIPTION OF THE INVENTION

### Polypeptide

This invention concerns an isolated polypeptide comprising or consisting of:

a) the TGKIINFIKFDTGNL sequence (SEQ ID NO: 1), or

b) a peptide fragment of at least 9 consecutive amino acids of the sequence, SEQ ID NO: 1, or

c) a variant of the sequence SEQ ID NO: 1 or of the fragment according to b), differing from the sequence SEQ ID NO: 1, or from the fragment according to b) by conservative substitution of one, two or three amino acids, or

d) a peptidomimetic of the sequence, SEQ ID NO: 1, of the fragment according to b) or of the variant according to c),

the said peptide fragment having the capacity to activate T-lymphocytes when it is presented by the HLA-DR7 molecule;

the said polypeptide not comprising the complete sequence of the protein encoded by the RPS4Y gene.

This invention concerns an isolated polypeptide comprising or consisting of:

a) the TGKIINFIKFDTGNL sequence (SEQ ID NO: 1), or

b) a peptide fragment of at least 9 consecutive amino acids of the sequence SEQ ID NO: 1, or

c) a variant of the sequence SEQ ID NO: 1 or of the fragment according to b), differing from the sequence SEQ ID NO: 1, or of the fragment according to b) by conservative substitution of one, two or three amino acids, or

d) a peptidomimetic of the sequence SEQ ID NO: 1, of the fragment according to b) or of the variant according to c),

the said peptide fragment, having the capacity to activate T-lymphocytes when it is presented by the HLA-DR7 molecule;

the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3).

In the context of the invention, the term “HLA” or “human leukocyte antigen” designates a human class I or class II Major Histocompatibility Complex (MHC), a gene cluster located on the short arm of chromosome 6. The MHC is divided into three regions: the class I region that contains the main HLA-A, HLA-B and HLA-C genes; the class II region that contains the HLA class II DR, DQ and DP genes; and the class III region that contains genes encoding other products that intervene in the immune response.

The HLA class II molecules are dimers consisting of a heavy a chain and a light 6 chain, both encoded in the MHC class II region. Each chain is organised in domains: two external N-terminal domains (α1, α2 and β1, β2 respectively), a hydrophobic transmembrane domain and an intracytoplasmic C-terminal domain. Each MHC class II locus—DR, DQ and DP—has two A and B genes that encode the corresponding a and 6 chains. The DRβ chain has the distinctive feature of being encoded by four loci (HLA-DRB1, HLA-DRB3, HLA-DRB4 and HLA-DRB5). However, not more than three functional loci are present in one individual, and not more than two on one chromosome. According to the HLA haplotype, one or two DR molecules are expressed: the αβ1 molecule, encoded by the DRA and DRB1 loci, is always present. Individuals who express haplotype DR3, DR17, DR18, DR5 (DR11, DR12), DR6 (DR13 or DR14) carry and express the DRB3 gene. DR4, DR7 or DR9 individuals carry and express the DRB4 gene, while DR2 (DR15 or DR16) individuals carry and express the DRB5 gene. DR1, DR10 or DR8 individuals only express the DRB1 gene.

As is well known by persons skilled in the art, the nomenclature for the HLA class II alleles, and in particular the HLA-DR alleles, is variable. Table 1 below shows the correspondence between the former and current nomenclature for the HLA-DR alleles. In the current nomenclature, the identification of the class II gene is followed by an asterisk, then the allele number, and finally that of the sub-allele. Therefore, the allele “HLA-DRB1*0701” corresponds to the DRB1 locus, allele 07, sub-type 01.

TABLE 1
Correspondence between the former and current nomenclature
for the HLA-DR alleles

| Current nomenclature | Former nomenclature |
|---|---|
| DRB1*0101 | DR1, Dw1 |
| DRB1*0102 | DR1, Dw20 |
| DRB1*0103 | DR′BR′, Dw′BON′ |
| DRB1*1501 | DR2, DRw15, Dw2 |
| DRB1*1502 | DR2, DRw15, Dw12 |
| DRB1*1601 | DR2, DRw16, Dw21 |
| DRB1*1602 | DR2, DRw16, Dw22 |
| DRB1*0301 | DR3, DRw17, Dw3 |
| DRB1*0302 | DR3, DRw18 |
| DRB1*0401 | DR4, Dw4 |
| DRB1*0402 | DR4, Dw10 |
| DRB1*0403 | DR4, Dw13, 13.1 |
| DRB1*0404 | DR4, Dw14, 14.1 |
| DRB1*0405 | DR4, Dw15 |
| DRB1*0406 | DR4, Dw′KT2′ |
| DRB1*0407 | DR4, Dw13, 13.2 |
| DRB1*0408 | DR4, Dw14, Dw14.2 |
| DRB1*0409 | DR4 |
| DRB1*0410 | DR4 |
| DRB1*0411 | DR4 |
| DRB1*1101 | DR5, DRw11, Dw5, DRw11.1 |
| DRB1*1102 | DR5, DRw11, DRw11.2 |
| DRB1*1103 | DR5, DRw11, DRw11.3 |
| DRB1*1104 | DR5, DRw11 |
| DRB1*1201 | DR5, DRw12, Dw′DB6′ |
| DRB1*1202 | DR5, DRw12, DRw12b |
| DRB1*1301 | DRw6, DRw13, Dw18, DRw6a |
| DRB1*1302 | DRw6, DRw13, Dw19, DRw6c |
| DRB1*1303 | DRw6, DRw13, Dw′HAG′ |
| DRB1*1304 | DRw6, DRw13 |
| DRB1*1305 | DRw6, DRw13 |
| DRB1*1401 | DRw6, DRw14, Dw9, Drw6b |
| DRB1*1402 | DRw6, DRw14, Dw16 |
| DRB1*1403 | DRw6, DRw14 |
| DRB1*1404 | DRw6, DRw6b.2 |
| DRB1*1405 | DRw6, DRw14 |
| DRB1*0701 | DR7, Dw17 |
| DRB1*0801 | DRw8, Dw8.1 |
| DRB1*08021 | DRw8, Dw8.2 |
| DRB1*08022 | DRw8, Dw8.2 |
| DRB1*08031 | DRw8, Dw8.3 |
| DRB1*08032 | DRw8, Dw8.3 |
| DRB1*0804 | DRw8 |
| DRB1*09011 | DR9, Dw23 |
| DRB1*09012 | DR9, Dw23 |
| DRB1*1001 | DRw10 |
| DRB3*0101 | DRw52a, Dw24 |
| DRB3*0201 | DRw52b, Dw25 |
| DRB3*0202 | DRw52b, Dw25 |
| DRB3*0301 | DRw52c, Dw26 |
| DRB4*0101 | DRw53 |
| DRB5*0101 | DR2, DRw15, Dw2 |
| DRB5*0102 | DR2, DRw15, Dw12 |
| DRB5*0201 | DR2, DRw16, Dw21 |
| DRB5*0202 | DR2, DRw16, Dw22 |

Therefore, the HLA-DR7 serotype is an HLA-DR serotype that recognizes gene products from DRB1*0701 to DRB1*0705 alleles.

The most polymorphic external domains of the HLA class II (α1 and β1) molecule form an open cavity on the sides, enabling a peptide of approximately 15 to 25 amino acids to be lodged. The peptide might interact more specifically on the cavity through three or four anchored residues located in the central part of the peptide whose side chains might plunge into the pockets at the bottom of the groove. Therefore, an optimal sequence peptide binds and is presented by a specific corresponding HLA class II molecule.

In the context of the invention, the term “peptide” is used interchangeably with the term “polypeptide” to designate a series of residues, typically L-amino acids, interconnected by peptide bonds between the α-amino and carboxyl groups of adjacent amino acids.

In some embodiments, the polypeptide of the invention consists of fewer than 50 amino acids, preferably fewer than 45 amino acids, fewer than 40 amino acids, fewer than 35 amino acids, fewer than 30 amino acids, fewer than 25 amino acids, fewer than 20 amino acids, and most preferably, fewer than 15 amino acids. More preferably, the polypeptide of the invention consists of 9 to 40 amino acids, preferably 10 to 39 amino acids, 11 to 38 amino acids, 12 to 37 amino acids, 13 to 36 amino acids, 14 to 35 amino acids, 15 to 34 amino acids, 16 to 33 amino acids, 17 to 32 amino acids, 18 to 31 amino acids, 19 to 30 amino acids, 20 to 29 amino acids, 21 to 28 amino acids, 22 to 27 amino acids, 23 to 26 amino acids, or 24 to 25 amino acids. In one preferred embodiment of the invention, the polypeptide consists of 40 amino acids. In another preferred embodiment, the polypeptide of the invention consists of 20, 19, 18, 17, 16, 15, 14, 13, 12, 11, 10 or 9 amino acids. In a particularly preferred embodiment, the polypeptide of the invention consists of 15 amino acids.

In one particular embodiment, the polypeptide of the invention comprises or consists of the sequence SEQ ID NO: 4.

A “peptidomimetic” of a reference peptide means herein a polypeptide that is chemically or enzymatically modified in comparison to the reference peptide, for example to improve its stability or bioavailability. The peptidomimetics of the invention can, in particular, be obtained by structural modification of the reference peptide, preferably by using unnatural amino acids such as D-amino acids instead of L-amino acids, in particular in the sequence SEQ ID NO: 1, thereby inducing a change in chirality, conformational restrictions, isosteric replacements, ring formation or other modifications. Other preferred modifications include those in which one or more amide bonds (is) are replaced by a non-amide bond or (is) are modified, for example, by acylation (preferably acetylation) or alkylation at the nitrogen or a carbon level; and/or one or more amino acid side chains (is) are replaced by a different chemical group; or one or more from the N-terminal extremity, the C-terminal extremity and one or more side chains (is) are protected by a protecting group, and/or double bonds and/or a ring formation and/or a stereospecificity is introduced in the amino acid chain to increase the rigidity and/or the binding affinity. The following are also envisaged: modifications in the C-terminal or the N-terminal extremity of the polypeptides such as deamination or N-terminal acylation (preferably acetylation), or such as amidation or C-terminal esterification; changes in azapeptides in which one or more a carbons (is) are replaced by nitrogen atoms; changes in β-peptides to which one or more carbons (is) are added on the N-α or C-α side of the main chain.

For this reason, one or more of the lysine amino acids (K) of the peptides can be modified, notably by amidation, amination, or N-oxide, N-nitroso, N-dialkyl phosphoryl, N-sulphenyl, or N-glycoside group formation.

In addition, the following can also or alternatively be modified: one or more threonine (T) and/or serine (S) amino acids of the polypeptides, notably by introducing on the OH group of the threonine and/or serine side chain, an ester or ether group. Esterification can be carried out with the use of a carboxylic acid, an anhydride, by bridging, etc., for example, to form acetates or benzoates. Etherification, which produces more stable compounds, can be carried out using an alcohol, a halide, etc., for example to form a methyl ether or an O-glycoside.

In addition, the following can also or alternatively be modified: one or more glutamine (Q) amino acids, for example, by amidation, by forming secondary or tertiary amines, notably, with groupings of the methyl or ethyl type, functionalized or not.

In addition, the following can also or alternatively be modified: one or more glutamate (E) and/or aspartate (D) amino acids, for example, by esterification to form methyl esters, substituted or not, ethyl esters, benzyl esters, thiols (activated esters); and by amidation, notably, to form N,N-dimethyl, nitroanilides, or pyrrolidinyl groups.

However, it is preferable not to modify proline amino acids that participate in the secondary structure of peptides, and it is further to be noted that in general, the G, A and M amino acids cannot be modified to any obviously useful end.

“Conservative substitution” means herein the replacement of one amino acid by another with similar properties (for example, polarity, hydrogen bonding potential, acidity, basicity, hydrophobicity, presence of an aromatic group, etc.). Amino acids with similar properties are well-known by persons skilled in the art. For example, arginine, histidine and lysine, which are basic hydrophilic amino acids, can be interchangeable. Similarly, isoleucine, a hydrophobic amino acid, can be replaced by leucine, methionine or valine.

Examples of conservative substitutions are presented in table 2 below.

TABLE 2
Conservative substitutions I

| Characteristics of the side chain | Amino acid |
|---|---|
| Non-polar | G A P I L V |
| Polar uncharged | C S T M N Q |
| Polar charged | D E K R |
| Aromatic | H F W Y |
| Other | N Q D E |

Alternatively, conservative amino acids can be grouped as described in Lehninger (1975: Biochemistry, 2nd Edition, Worth Publishers, Inc. New-York: NY., p. 71-77), as shown in table 3 below.

TABLE 3
Conservative substitutions II

| Characteristics of the side chain | | Amino acid |
|---|---|---|
| Non-polar | Aliphatic | A L I V P |
| | Aromatic | F W |
| | Sulphur containing | M |
| | Borderline | G |
| Polar uncharged | Hydroxyl | S T Y |
| | Amides | N Q |
| | Sulphydryl | C |
| | Borderline | G |

| | |
|---|---|
| Positively charged (basic) | K R H |
| Negatively charged (acid) | D E |

According to another alternative, examples of conservative substitutions are presented in table 4 below.

TABLE 4
Conservative substitutions III

| Original residue | Example of substitution |
|---|---|
| A | V L I |
| R | K Q N |
| N | Q H K R |
| D | E |
| C | S |
| G | N |
| E | D |
| H | N Q K R |
| I | L V M A F |
| L | I V M A F |
| K | R Q N |
| M | L F I |
| F | L V I A |
| P | G |
| S | T |
| T | S |
| W | Y |
| Y | W F T S |
| V | I L M F A |

By “isolated” polypeptide is meant herein a polypeptide isolated from the human body or from the organism of a non-human mammal. In particular, an isolated polypeptide according to the invention is preferably one that is substantially or essentially rid of components by which it is usually accompanied when found in its native state. Therefore, the peptides of the invention do not comprise materials that are normally associated with them in their in situ environment, such as HLA class II molecules on antigen-presenting cells. However, the isolated polypeptide may, for example, be present in a pharmaceutical composition or in a kit. The polypeptide is preferably present in one of the pharmaceutical compositions described below. This polypeptide is preferably in a purified form.

By “fragment” of a reference sequence is meant herein a sequence that is smaller than the reference sequence. In the contexte of this invention, peptide fragments are at least 9 amino acids long. They may, for example, be 9, 10, 11, 12, 13, or 14 amino acids long.

By “peptide fragment of at least x amino acids” is meant herein that the peptide fragment consists of a sequence of at least x amino acids.

The polypeptides of the invention have the capacity to activate T-lymphocytes when they are presented by the HLA-DR7 molecule.

By “polypeptide presented by the HLA-DR7 molecule” is meant herein that the polypeptide is linked to the HLA-DR7 molecule in such a manner that it can also be recognized by a receptor present on the surface of T-lymphocytes.

The techniques that make it possible to show the binding between a peptide and an HLA molecule are well known by persons skilled in the art. They can, for example, implement live cells as described in Ceppellini et al. (1989) Nature 339:392; Christinck et al. (1991) Nature 352:67; Busch et al. (1990) Int. Immunol. 2:443; Hill et al. (1991) J. Immunol. 147:189 or del Guercio et al. (1995) J. Immunol. 154:685; acellular systems that use lysates obtained with detergents, as described in Cerundolo et al. (1991) J. Immunol. 21:2069; purified immobilised HLA molecules as described in Hill et al. (1994) J. Immunol. 152: 2890 or Marshall et al. (1994) J. Immunol. 152: 4946; ELISA systems as described in Reay et al. (1992) EMBO J. 11:2829; surface plasmon resonance as described in Khilko et al. (1993) J. Biol. Chem. 268: 15425; or high flow soluble phase assays (Hammer et al. (1994) J. Exp. Med. 180: 2353). The binding between a peptide and an HLA-DR molecule is typically detected by use of a protocol that consists of inducing recognition of the HLA-peptide complex by T-lymphocytes that express a T receptor specific to this complex. This recognition can be measured by techniques that are well-known by persons skilled in the art, such as techniques of measurement of cell proliferation if the T-lymphocyte carries out a helper function; of measurement of antigen-presenting cell lysis if the T-lymphocyte is cytotoxic; of measurement of interleukin secretion, of measurement of the level of the mRNAs that encode these interleukins, or of measurement of the activation of the biochemical cascade that leads to the activation of transcription factors.

By “capable of activating T-lymphocytes” is meant herein that when it is presented by the HLA-DR7 molecule, the polypeptide is capable of binding to the T-lymphocyte TCR in the form of a trimolecular complexe involving the HLA α and β chains, and then inducing, via a signalling cascade, the expression by the lymphocyte of immediate (such as c-fos, NFAT-1, c-myc and/or NF-κB), early (such as the cytokines IFN-γ, IL-2, TGF-β, IL-10, FoxP3, IL-4, IL-5, IL13 and/or GM-CSF, the insulin receptors R, IL-2R and/or transferrin receptor R, the activation molecules CD69 or CD25, and/or the intracellular proteins ODC, actin, cyclin, transferrin and/or histones), and/or late (such as the MHC HLA-DR, the cytokine Rantes, and/or the activation proteins VLA-4 and/or VLA-1) genes, and/or chemokine receptors, and/or selectin ligands such as CD62L.

Techniques through which the activation of T-lymphocytes by a polypeptide can be demonstrated are well-known by persons skilled in the art and include for example, mixed lymphocyte reaction (MLR) assays as described in the example below; determination of cytokine secretion by T-lymphocytes, in particular IL-4, IL-10, IL-2 and/or IFNγ cytokines, for example by using ELISA assays, or measurement of the cytokine that encodes messenger RNAs encoding these cytokines; or cell division monitoring with an intracellular marker such as CFSE.

The polypeptides of the invention neither comprise the complete sequence of the protein encoded by the RPS4Y gene, nor the sequence SEQ ID NO: 2, nor the sequence SEQ ID NO: 3.

By “RPS4Y” or “ribosomal protein S4 Y-linked” gene is meant herein a gene that encodes the small sub-unit 4 of the ribosomal protein, a protein involved in mRNA binding and located on the interface of sub-units 40S/60S of the small ribosomal sub-unit. The RPS4Y gene is located on the Y chromosome. In the context of the invention, the RSP4Y gene is the human RPS4Y gene. Typically, the complete sequence of the protein encoded by the RPS4Y gene consists of 263 amino acids and is preferably represented by the sequence SEQ ID NO: 5.

The sequence SEQ ID NO: 2 is a fragment of the protein RPS4Y, described in Ivanov et al. (2005) Clin. Cancer Res. 11:1694-1703, identified as being presented by the HLA class I HLA-B*5201 molecule, which is an HLA allele that is very rare in the general population.

The sequence, SEQ ID NO: 3 is a fragment of the protein RPS4Y, described in Spierings et al. (2003) Lancet 362:610-615, identified as being presented by the HLA class II HLA-DRB3*0301 molecule.

Preferably, the polypeptide of the invention consists of the sequence TGKIINFIKFDTGNL (SEQ ID NO: 1). The polypeptide of the invention may also consist of a sequence selected from the group consisting of the sequence TGKIINFIK (SEQ ID NO: 6), the sequence GKIINFIKF (SEQ ID NO: 7), the sequence KIINFIKFD (SEQ ID NO: 8), the sequence IINFIKFDT (SEQ ID NO: 9), the sequence INFIKFDTG (SEQ ID NO: 10), the sequence NFIKFDTGN (SEQ ID NO: 11) and the sequence FIKFDTGNL (SEQ ID NO: 12).

### Nucleic Acid and Vector

This invention also concerns a nucleic acid comprising or consisting of a sequence that encodes a polypeptide as defined above.

Such a nucleic acid can easily be obtained by cloning cDNA fragments that encode the protein RPS4Y.

This nucleic acid can be inserted in an expression vector in which it is operatively linked to one or more elements that enable its expression or the regulation of its expression, notably, such as transcriptional promoters, activators and/or terminators.

The signals that control the expression of nucleotide sequences (promoters, activators, termination sequences, etc. . . . ) are chosen according to the cell host used. To that end, the nucleic acids of the invention can be inserted in autonomously replicating vectors within the chosen host, or integrating vectors of the chosen host. Such vectors will be prepared according to methods that are commonly used by persons skilled in the art, and the resulting clones can be introduced in an appropriate host by standard methods such as electroporation or calcium phosphate precipitation.

The cloning and/or expression vectors as described above, that contain a nucleic acid defined according to the invention are also a part of this invention.

Moreover, the invention concerns host cells transfected transiently or stably, by these expression vectors. These cells can be obtained by introducing, into the prokaryote or eukaryote host cells, a nucleotide sequence inserted in a vector as defined above, and then culturing the said cells under conditions that enable the replication and/or the expression of the transfected nucleotide sequence.

Examples of host cells include notably, human cells such as HEK293, PER.C6, non-human mammalian cells such as CHO, COS, MDCK, insect cells such as SF9, bacteria such as Escherichia coli, and fungal and/or yeast strains such as L40 and Y90.

### Polypeptide Production Method

This invention also concerns a method for producing a polypeptide as defined above, comprising the steps that consist of:

a) chemically or enzymatically synthesizing the said polypeptide or inducing the expression of the said polypeptide by a cell comprising the nucleic acid or the vector as defined above; and

b) retrieving the said polypeptide obtained in step a).

More generally, the polypeptides that are useful in this invention can be synthesized by any well-known method used by persons skilled in the art. Such methods include notably, standard chemical synthesis (in solid phase or in liquid homogeneous phase), enzymatic synthesis from constitutive amino acids or their derivatives, as well as biological production methods by recombinant host cells.

Chemical synthesis is particularly advantageous due to purity, antigen specificity, absence of undesired secondary products reasons and for the ease of production. Therefore, an object of the invention is a method for producing a polypeptide of the invention comprising the chemical synthesis of the said polypeptide. The polypeptide obtained can then be optionally purified by any method well-known by persons skilled in the art. The production method can also include one or more steps of chemical or enzymatic modification of the polypeptide to improve its stability or bioavailability, as well as one or more steps to bind the polypeptide to a therapeutic compound.

Chemical synthesis includes, among other methods, Merrifield type synthesis and Fmoc solid phase peptide synthesis (see for example, “Fmoc solid Phase peptide synthesis, a practical approach”, published by W. C. Chan and P. D. White, Oxford University Press, 2000).

The method for producing the polypeptide according to the invention can, in addition, include a step of formulation of the obtained polypeptide in a pharmaceutical composition, for example, one of the compositions described in the section below.

Another object of the invention is a method of biological production of a polypeptide according to the invention, by a recombinant host cell. In such a method, a vector containing a nucleic acid encoding a polypeptide of the invention is transferred to a host cell that is cultured under conditions that enable the expression of the corresponding polypeptide.

The polypeptide that is produced can then be retrieved and purified.

The purification methods used are known by persons skilled in the art. The recombinant polypeptide obtained can be purified from cell lysates and extracts, from the supernatant of the culture medium, by methods used individually or in combination, such as fractionation, chromatography methods, immunoaffinity techniques by use of specific monoclonal or polyclonal antibodies, etc.

### T-Lymphocyte

In addition, this invention concerns an isolated T-lymphocyte capable of specifically recognizing, or that specifically recognizes, an epitope from a polypeptide as defined above or a polypeptide comprising the complete sequence of the protein encoded by the RPS4Y gene and presented by the HLA-DR7 molecule expressed on the surface of antigen-presenting cells.

By “T-lymphocyte” is meant herein an immune system cell displaying a CD3 receptor on its surface, and which is involved in cell immunity. As is well-known to persons skilled in the art, T-lymphocytes include cytotoxic T-lymphocytes, helper T cells, regulatory T cells, NKT cells and Tγδ lymphocytes.

Cytotoxic T lymphocytes, CTL or CD8⁺ T-lymphocytes destroy infected cells that display a specific antigen that they recognize.

T helper cells, T helpers or Th are T-lymphocytes displaying the CD4 receptor on their surface and which proliferate to activate other types of immune system cells.

Regulatory T cells or Tregs are T-lymphocytes displaying both CD4 and CD25 receptors on their surface and expressing the protein FOXP3 in their cytosol when they are in a basal state. They suppress the activity of immune cells; either the autoimmune activity, or the activity at the end of immune reactions.

NKT cells are lymphocytes displaying both the CD3 marker and NK cells markers. They recognize a glycolipid presented by the CD1d molecule, thereby enabling their activation.

Tγδ lymphocytes are T-lymphocytes with a TCR consisting of a γ chain and a δ chain, and are present in large quantities in intestinal mucosa.

Preferably, the isolated T-lymphocyte of the invention is a CD8⁺ and/or CD4⁺ T-lymphocyte. More preferably, the isolated T-lymphocyte of the invention is a CD4⁺ T-lymphocyte.

In the context of the invention, the expression “T-lymphocyte capable of specifically recognizing” means that the T-lymphocyte is capable of binding the amino acid sequence of an epitope from the polypeptide of the invention or a polypeptide comprising the complete sequence of the protein encoded by the RPS4Y gene and presented by the HLA-DR7 molecule expressed on the surface of antigen-presenting cells, but is incapable of binding an epitope of another amino acid sequence which is presented by an HLA molecule other than the HLA-DR7 molecule on the surface of antigen-presenting cells.

When the term “specifically” refers to a T-lymphocyte recognition or binding specific for an antigen, it means that the T-lymphocyte interacts with the antigen without substantial interaction with other antigens, or if it concerns “specific” recognition with an epitope, means almost exclusive recognition of this epitope.

As is well-known to persons skilled in the art, the epitopes presented by HLA class II molecules are derived from polypeptides that are internalised and degraded before becoming attached to HLA molecule peptide binding sites, so that the T-lymphocytes can recognize them. More particularly, polypeptides are internalised by endocytosis or after binding to a receptor surface, and are then degraded by proteases of the acidic endolysosomal compartment into epitopic peptides of about twenty amino acids. In parallel, the neosynthesized HLA class II molecules (α and β chains) are assembled in the endoplasmic reticulum where they are stabilised in the presence of the invariant chain (Ii). Nonameric complexes (3 invariant (Ii) chains, 3 α chains and 3β chains) migrate through the Golgi and are directed towards the MIIC endosomal compartment. During this migration, the peptide binding site of the HLA class II molecules is constantly occupied by the CLIP sequence of the invariant (Ii) chain which maintains the cavity in its right conformation. Once inside the acidic endolysosomal compartment, the invariant (Ii) chain is degraded and the CLIP peptide is released. The HLA class II molecules then bind to peptides from the endosomal proteolysis, and the class II molecules displaying these peptides then migrate to the cell surface.

Consequently, by “epitope from a polypeptide and presented by the HLA-DR7 molecule expressed at the surface of antigen-presenting cells” is meant herein a peptide produced after internalisation and degradation of the said polypeptide by proteases in an antigen-presenting cell, and capable of binding, in the acidic endolysosomal compartment of the said antigen-presenting cell, with an HLA-DR7 molecule the peptide binding site of which is free. Preferably, the epitope of the invention consists of a sequence of 9 to 25 amino acids, preferably 10 to 24 amino acids, preferably 11 to 23 amino acids, preferably 12 to 22 amino acids, preferably 13 to 21 amino acids, preferably 14 to 20 amino acids. Even more preferably, the epitope of the invention consists of a sequence of 15 amino acids. Still preferably, the epitope of the invention consists of a sequence of 9 amino acids.

The inventors have indeed shown that the polypeptide of the invention binds specifically to T-lymphocytes when it is presented by the HLA-DR7 molecule.

Consequently, preferably the isolated T-lymphocyte of the invention is capable of specifically recognizing or recognizes specifically an epitope from the polypeptide consisting of the sequence SEQ ID NO: 1 when this epitope is presented by the HLA-DR7 molecule.

Preferably, the isolated T-lymphocyte of the invention is likely to be obtained, or is obtained by the T-lymphocyte preparation method of the invention as defined in the section “T-lymphocyte preparation method” below.

### T-Lymphocyte Preparation Method

This invention also concerns a method of in vitro preparation of T-lymphocytes capable of specifically recognizing an epitope from a polypeptide of the invention or a polypeptide comprising the complete sequence of the protein encoded by the RPS4Y gene and presented by the HLA-DR7 molecule expressed on the surface of antigen-presenting cells, comprising the steps of:
T-lymphocyte co-culture with antigen-presenting cells that express on their surface the HLA-DR7 molecule to which is attached an epitope from the polypeptide of the invention or a polypeptide comprising the complete sequence of the protein encoded by the RPS4Y gene; and
retrieval of T-lymphocytes from the said co-culture.

The term “antigen-presenting cell” or “APC” means herein a cell that typically expresses an HLA class I or II molecule on its surface. Preferably, the antigen-presenting cell of the invention expresses an HLA class II molecule on its surface, and even more preferably, an HLA-DR molecule, and most preferably, the HLA-DR7 molecule.

Antigen-presenting cells can be of monocyte or macrophage, B-lymphocyte, and dendritic cell types. Preferably, the antigen-presenting cell of the invention is a dendritic cell. Even more preferably, the antigen-presenting cell of the invention is from a female subject. Preferably, the antigen-presenting cell of the invention and the T-lymphocyte with which it is co-cultured are HLA-identical.

The antigen-presenting cells used in the method of the invention, express on their surface the HLA-DR7 molecule to which is attached an epitope from the polypeptide of the invention or a polypeptide comprising the complete sequence of the protein encoded by the RPS4Y gene.

Such antigen-presenting cells can be obtained by loading the cells with the polypeptide of the invention or with a polypeptide comprising the complete sequence of the protein encoded by the RPS4Y gene, as described in Mutis et al. (1999) Blood 93:2336-2341. Typically, the antigen-presenting cells are pulsed with the polypeptide of the invention or with a polypeptide comprising the complete sequence of the protein encoded by the RPS4Y gene, preferably at a concentration of about 10 μg/ml, for 90 minutes, at a temperature of 37° C. in a culture medium without serum, for example.

These antigen-presenting cells can also be obtained by transfection or transduction with a vector comprising a nucleic acid that encodes a polypeptide of the invention or that encodes a polypeptide comprising the complete sequence of the protein encoded by the RPS4Y gene, as described for example in Mutis et al. (2002) Biol. Blood Marrow Transplant. 8:412-419.

The epitope from the polypeptide of the invention or from a polypeptide comprising the complete sequence of the protein encoded by the RPS4Y gene and presented by the HLA-DR7 molecule, will then be produced by proteolytic degradation, in the acidic endolysosomal compartment of the antigen-presenting cell, and then binding in this compartment with the said HLA-DR7 molecule whose peptide binding site would have be freed.

Preferably, the epitope of the invention consists of a sequence of 9 to 25 amino acids, preferably 10 to 24 amino acids, preferably 11 to 23 amino acids, preferably 12 to 22 amino acids, preferably 13 to 21 amino acids, preferably 14 to 20 amino acids. Even more preferably, the epitope of the invention consists of a sequence of 15 amino acids. Still preferably, the epitope of the invention consists of a sequence of 9 amino acids.

The step of co-culturing the T-lymphocytes with the antigen-presenting cells takes place preferably in an appropriate culture medium supplemented with human AB serum, typically in RPMI medium supplemented with serum, and in the presence of IL-2, preferably at a temperature of about 37° C. In addition, the T-lymphocytes can be put back in culture several times with new antigen-presenting cells as defined above, preferably every 10 to 14 days.

### Pharmaceutical Composition and Therapeutic Uses

This invention also concerns a pharmaceutical composition comprising (i) a polypeptide as defined above or a T-lymphocyte as defined above and (ii) a pharmaceutically acceptable vehicle.

This invention also concerns a medicament comprising a polypeptide as defined above or a T-lymphocyte as defined above. It also concerns a method for treating a subject, in which a therapeutically effective quantity of the polypeptide of the invention or of the T-lymphocyte of the invention is administered to the said subject in need thereof. It also concerns the use of the polypeptide or the T-lymphocyte of the invention for the manufacture of a medicament.

“Treatment” indicates a remedial treatment (aiming at least at relieving, slowing down or stopping the development of the disease).

“Excipient” or “pharmaceutically acceptable vehicle” indicates any solvent, dispersion medium, absorption delaying agent, etc., that does not produce any secondary reaction, for example, allergic, in humans or animals.

In case of the use of T-lymphocytes of the invention, non-restrictive examples of pharmaceutically acceptable vehicles include notably, a physiological solution, i.e. with the same osmolarity as blood, and which can be a solution of double-distilled water containing 0.9 g/l of NaCl, or a Ringer's or Ringer's lactate solution. The T-lymphocytes of the invention can be put in suspension, for example, in a total volume of 10, 50, 100, 150, 200, 250, 300, 400, or 500 ml of physiological solution.

Pharmaceutical compositions and medicaments of the invention can be formulated so that they can be administered to patients by a single route or by different routes.

When administration by parenteral route is envisaged, more specifically by injection, the compositions of the invention are in the form of solutes and injectable suspensions packaged in ampoules or vials for slow infusion. The injection can notably be administered by subcutaneous, intramuscular or intravenous route.

In case of administration by oral route, the compositions of the invention are in the form of capsules, effervescent tablets, bare or coated tablets, pouches, sugar-coated tablets, drinkable ampoules or solutes, micro-granules or prolonged-release forms.

The forms for parenteral administration are obtained in a standard manner by mixing the active ingredient(s) with buffers, stabilising agents, preservatives, solubilising agents, isotonic agents and suspending agents. In conformance with well-known techniques, these mixtures are then sterilised and then packaged in the form of intravenous injections.

As buffers, persons skilled in the art could use organic phosphate salt-based buffers.

Examples of suspending agents include methylcellulose, hydroxyethylcellulose, hydroxypropylcellulose, acacia and sodium carboxymethylcellulose.

In addition, useful stabilisers of the invention are sodium sulphite and sodium metasulphite, while sodium p-hydroxybenzoate, sorbic acid, cresol, and chlorocresol can be cited as preservatives. For the preparation of oral solution or suspension, the active ingredients are dissolved or put in suspension in an appropriate vehicle with a dispersing agent, a wetting agent, a suspending agent (for example, polyvinylpyrrolidone), a preservative (such as methylparaben or propylparaben), a flavouring agent or a dye.

For the preparation of microcapsules, the active ingredients are combined with appropriate diluents, appropriate stabilisers, agents that promote the prolonged-release of active substances or any other type of additive for the formation of a central core that is then coated with an appropriate polymer (for example, a water-soluble resin or a resin that is insoluble in water). The techniques known by persons skilled in the art will be used for this purpose.

Microcapsules thereby obtained might then be formulated in appropriate unit doses.

The peptides of the invention can also be formulated in the form of liposomes. Liposomes are formed from phospholipids which are dispersed in an aqueous medium and spontaneously form multilamellar concentric bilayer vesicles. These vesicles usually have a diameter of 25 nm to 4 μm and can be sonicated, leading to the formation of smaller unilamellar vesicles with a diameter of 200 to 500 Å, containing an aqueous solution in their core.

Liposomes can be particularly advantageous for administering the medicament to a precise target cell or tissue. To this end, the lipids can be chemically coupled with targeting molecules or ligands, such as targeting peptides (for example, hormones) or antibodies. In particular, to target immune cells, a ligand to be incorporated into the liposome can, for example, be an antibody or antibody fragment specific of cell surface determinants of the targeted immune system cells.

This invention also concerns more specifically, an isolated polypeptide as defined above, or an isolated T-lymphocyte as defined above, or a polypeptide comprising the complete sequence of the protein encoded by the RPS4Y gene, for use in the treatment of cancer of immune cells.

It also concerns the use of an isolated polypeptide as defined above, or an isolated T-lymphocyte as defined above, or a polypeptide comprising the complete sequence of the protein encoded by the RPS4Y gene, for the manufacture a medicament intended to treat cancer of immune cells.

It also concerns a method for treating cancer of immune cells in which a therapeutically effective quantity of an isolated polypeptide as defined above, or of an isolated T-lymphocyte as defined above, or of a polypeptide comprising the complete sequence of the protein encoded by the RPS4Y gene, is administered to a patient in need thereof.

Cancers of immune cells are well-known by persons skilled in the art and include, in particular, leukaemias and lymphomas. The cancer of immune cells can therefore be chosen from among acute lymphoblastic leukaemia (ALL), acute myeloid leukaemia (AML), chronic lymphoid leukaemia (CLL) and chronic myeloid leukaemia (CML). The cancer of immune cells can also be chosen from Hodgkin's lymphoma and non-Hodgkin's lymphoma. The cancer of immune cells can also be a relapse of a previous cancer, in particular, a relapse of leukaemia.

Preferably, the treated subject is a human male subject. Preferably, the treated subject expresses the HLA-DR7 molecule. Even more preferably, the treated subject displays the HLA-DRB1*0701 allele.

More preferably, this invention therefore concerns an isolated polypeptide as defined above, or an isolated T-lymphocyte as defined above, or a polypeptide comprising the complete sequence of the protein encoded by the RPS4Y gene, for use in the treatment of a cancer of immune cells in a subject who expresses the HLA-DR7 molecule.

Preferably, the subject has been treated beforehand by chemotherapy and/or radiation therapy. Preferably, the treated subject has also had bone marrow irradiation. Without willing to be bound by a mechanism of action, the administered polypeptide will then induce lysis or activate an immune response vis-à-vis the cells of the subject to be treated, in particular vis-à-vis male cells that express the HLA-DR7 molecule. Indeed, after chemotherapy and/or irradiation treatment, these cells will be almost exclusively cancer cells since non-cancer cells that express the HLA-DR7 molecules are mainly haematopoietic cells that would have been eliminated by chemotherapy and/or irradiation.

Preferably, when the polypeptide of the invention or the polypeptide comprising the complete sequence of the protein encoded by the RPS4Y gene is administered, the subject can also undergo or have further undergone a bone marrow transplant, preferably from a female and/or HLA-identical donor. In fact, without willing to be bound by a mechanism of action, this transplantation will enable the reconstitution of the bone marrow of the treated subject, while enabling the specific recognition of residual or reappearing cancer cells, thanks to the polypeptide of the invention that targets cells that express the HLA-DR7 molecule.

Similarly, when T-lymphocytes of the invention are administered, the subject, in particular the subject suffering from a leukaemia relapse, can undergo or have undergone a bone marrow transplant, preferably a bone marrow transplant from a female donor and/or an HLA-identical donor. In particular, in this case, the subject may not have had bone marrow irradiation. In fact, without willing to be bound by theory, in this case, since the patient's haematopoietic cells are female further to the bone marrow transplant, only the male cells that express the HLA-DR7 molecules, i.e. cancer cells, will be targeted by the administered T-lymphocytes.

By “HLA-identical donor” is meant herein a subject carrying the same alleles of the HLA class I and HLA class II genes that encode the major HLAs, as the subject to be treated.

Preferably, the T-lymphocyte used in the treatment methods defined above is a heterologous T-lymphocyte, i.e. a T-lymphocyte that is not from the subject to be treated, but is preferably HLA-identical to the subject to be treated. The T-lymphocyte used in the treatment methods defined above can also be a non-HLA-identical heterologous T-lymphocyte but which expresses the HLA-DR7 molecule. Preferably, in this case, the T-lymphocyte is of clonal type and specifically recognizes the RPS4Y-HLA-DR7 peptide complex. This is in particular very useful in case of emergency for the subject to be treated because the immediate identification of an HLA-identical donor is not required.

By “therapeutically effective quantity” is meant a quantity that enabling relieving, slowing down or stopping the development of the disease in the subject to whom this quantity is administered.

An effective quantity for obtaining a therapeutic effect will depend, for example, on the peptide composition, the route of administration, the stage and severity of the disease to be treated, the weight and general health status of the patient and the opinion of the physician in charge of the case. Typically, the polypeptide or peptide fragment of the invention is administered at a dose of about 1.0 μg to about 5,000 μg for a patient weighing 70 kg.

The following examples and figures illustrate the invention without limiting its scope.

### BRIEF DESCRIPTION OF THE FIGURES

FIG. 1 presents histograms showing the level of proliferation (represented by the incorporation of ³HT in c.p.m×10⁻³ on day 3) of CD4/CD8 DP (black lines) or CD4⁺ single-positive (white lines) T-lymphocytes after stimulation with donor (D), or receiver (R) EBV-B-LCL, or HLA-B*1401 (B*1401) or HLA-DRB1*0701 (DRB1*0701).

FIG. 2 presents histograms showing the ratio of (A) the expression of IFNγ mRNA in relation to that of CD3, (B) the expression of FoxP3 mRNA in relation to that of CD3, (C) the expression of IL-4 mRNA in relation to that of CD3, (D) the expression of IL-10 mRNA in relation to that of PPIB, (E) the expression of TGFβ mRNA in relation to that of PPIB, by CD4/CD8 DP (CD4/CD8) T-lymphocytes or CD4⁺ single-positive T-lymphocytes (CD4) after 24 hours of stimulation with donor EBV-B-LCL (hatched diagonal lines descending to the right), HLA-B*1401 (tight hatched diagonal lines ascending to the right) or HLA-DRB1*0701 (hatched diagonal lines ascending to the right).

FIG. 3 presents histograms showing the level of proliferation (represented by the incorporation of BRDu) of the CD4⁺ T-lymphocyte clone after stimulation with female EBV-B-LCL that express HLA-DRB1*0701 (DR7 ♀) and if appropriate transfected with the DDX3Y (DDX3Y) or RPS4Y (RSPAY) genes or with male EBV-B-LCL that express HLA-DRB1*0701 (DR7 ♂).

FIG. 4 presents histograms showing the level of secretion of IFNγ (in μg/ml) by the CD4⁺ T-lymphocyte clone, non-stimulated (−) or after 24 hours of stimulation with female EBV-B-LCL that express HLA-DRB1*0701 (DR7 ♀) and if appropriate, transfected with the DDX3Y (DDX3Y) or RPS4Y (RSPAY) genes or with male EBV-B-LCL that express HLA-DRB1*0701 (DR7 ♂).

FIG. 5 presents histograms showing the level of secretion of IFNγ (in μg/μl) by the CD4⁺ T-lymphocyte clone after stimulation with female EBV-B-LCL that express HLA-DRB1*0701 (DR7 ♀) and if appropriate, transfected with the RPSA4Y (RPS4Y) gene or that express SK-31 (SK-31), KM-25 (KM-25), DA-32 (DA-32), EV-44 (EV-44), QR-40 (QR-40) or SG-41 (SG-41) peptides or with male EBV-B-LCL that express HLA-DRB1*0701 (DR7 ♂).

FIG. 6 presents histograms showing the level of secretion of IFNγ (in μg/μl) by the CD4⁺ T-lymphocyte clone, non-stimulated (CD4) or after stimulation of female EBV-B-LCL that express HLA-DRB1*0701 (DR7) in the presence or absence of QR-40 or TL15 peptides at the indicated concentration.

### DESCRIPTION OF SEQUENCES

| SEQ | |
|---|---|
| ID NO: | description |
| 1 | Peptide of sequence TGKIINFIKFDTGNL (also called peptide |
| | TL15) |
| 2 | Peptide of sequence TIRYPDPVI, excluded from the invention |
| 3 | Peptide of sequence VIKVNDTVQI, excluded from the invention |
| 4 | Peptide QR-40 |
| 5 | Protein encoded by the RPS4Y gene |
| 6 | First fragment of 9 consecutive amino acids of SEQ ID NO: 1 |
| 7 | Second fragment of 9 consecutive amino acids of SEQ ID NO: 1 |
| 8 | Third fragment of 9 consecutive amino acids of SEQ ID NO: 1 |
| 9 | Fourth fragment of 9 consecutive amino acids of SEQ ID NO: 1 |
| 10 | Fifth fragment of 9 consecutive amino acids of SEQ ID NO: 1 |
| 11 | Sixth fragment of 9 consecutive amino acids of SEQ ID NO: 1 |
| 12 | Seventh fragment of 9 consecutive amino acids of SEQ ID NO: 1 |
| 13 | Peptide SK-31 |
| 14 | Peptide KM-25 |
| 15 | Peptide DA-32 |
| 16 | Peptide EV-44 |
| 17 | Core motif from the Hotspot database, source of peptide SK-31 |
| 18 | Core motif from the Net MHC database, source of peptide SK-31 |
| 19 | Core motif from the Propred database, source of peptide SK-31 |
| 20 | Core motif from the Hotspot database, source of peptide KM-25 |
| 21 | Core motif from the Hotspot database, source of peptide DA-32 |
| 22 | Second core motif from the Hotspot database, source of peptide |
| | DA-32 |
| 23 | Core motif from the Hotspot database, source of peptide EV-44 |
| 24 | Core motif from the Net MHC database, source of peptide EV-44 |
| 25 | Core motif from the Propred database, source of peptide EV-44 |
| 26 | Second core motif from the Propred database, source of peptide |
| | EV-44 |
| 27 | Core motif from the Hotspot database, source of peptide QR-40 |
| 28 | Second core motif from the Hotspot database, source of peptide |
| | QR-40 |
| 29 | Core motif from the Net MHC database, source of peptide QR-40 |
| 30 | Core motif from the Propred database, source of peptide QR-40 |
| 31 | Peptide GV17 |
| 32 | Peptide TG17-1 |
| 33 | Peptide TG17-2 |

### Example

This example shows the identification by the inventors of the polypeptide of the invention

### Materials and Methods

### Medium and Reagents

RPMI-1640 medium (Life Technologies, Eggenstein, Germany) was supplemented with L-glutamine (2 mM), penicillin (100 UI/ml), streptomycin (100 μg/ml), NaHCO₃ (1.5 mg/ml) and 10% human AB serum inactivated by pooled heat. Recombinant human GM-CSF, rhIL-2, rhIL-4 and rhTNF-α were bought at R&D Systems (Abingdon, United Kingdom). HLA typing was evaluated by serology and characterised in depth by oligonucleotide typing. Anti-HLA class I (W632), anti-HLA-DR (L243) and anti-HLA-DP (B7-21) antibodies were supplied free of cost by J. Chopin (Hopital Cochin, ICGM, France), and the anti-HLA-DQ (SPVL3) antibody was bought at Immunotech (Marseille, France).

### Generation of Minor H Antigen-Specific T-Lymphocyte Lines and Clones

A skin biopsy harvested from a patient suffering from GVHD, 1 month post-transplantation, was cultured for 2 weeks in the presence of 20 IU/ml of IL-2 in a complete RPMI medium. This patient received a bone marrow transplant from his HLA-identical sister. His HLA typing was the following: A*0205/*6801; B*1401/*4403; C*0802:*1601; DRB1*0701; DQB1*0202; DPB1*0401/1101. The skin T-lymphocytes were phenotyped and propagated by several cycles of stimulation with APCs from the receiver, and cultured for 2 weeks in the presence of 20 IU/ml of IL-2. The APCs that were initially used were peripheral blood mononuclear cells (PBMC) from the receiver irradiated with 30 Gy, and then, after the third cycle, B-lymphocytes immortalised with the Epstein-Barr virus (EBV-B-LCL) of the receiver were used. They were irradiated with 50 Gy. This protocol resulted in T-lymphocyte cell lines.

Clones were derived from these cell lines, as previously described. In brief, specific T-lymphocytes were cloned by limit dilution to cell concentrations of 4, 1, or 0.4 cells/well, in round bottom microtiter plates with 96 wells (Falcon; Becton Dickinson, Mountain View, Calif., United States) in the presence of 2.5×10⁵/ml of irradiated PBMC (30 Gy) plus 0.5×10⁵/ml of irradiated EBV-B LCL (50 Gy) derived from the receiver and 10 U/ml of rIL-2. The T-lymphocyte clones that were generated were propagated in the presence of 0.5×10⁵ irradiated EBV-B LCL from the receiver, and rIL-2. The phenotype of the generated clones was analysed by FACScan using antibodies (CD4 and CD8) labelled by PE/FITC (BD Biosciences, Le Pont de Claix, France).

### Mixed Lymphocyte Reaction (MLR) Assays

1×10⁴ T-lymphocyte cell lines or clones were incubated in triplicate with 3×10⁴ EBV-B LCL irradiated by 50 Gy in a final volume of 200 μl in round bottom microtiter plates with 96 wells. After 3 days in culture, 1 μCi/well of ³H-[methyl-thymidine] was added during the last 16-18 h. The cells were collected by using a Filtermate 196 multiple harvester (Packard Inc. Prospect, Conn., United States) and the incorporation of thymidine was measured in a TopCount liquid scintillation counter (Packard, Inc.).

### Modified Assay of the Helper T-Lymphocyte Precursor (HTLP) Frequency

Numbers in gradient of PBMC responders were stimulated with 3×10⁴ receiver's EBV-BLCL, irradiated by 50 Gy. The cells were cultivated in round bottom microtiter plates with 96 wells in a final volume of 200 μl of culture medium and 10% human AB serum inactivated by heat. 16 replicates and 4 dilutions (10⁴, 5×10³, 2.5×10³, 1.5×10³) were made. CD4/CD8 DP T-lymphocytes were added or omitted in two different Treg/effector ratios: 1:4 or 1:1. 24 h hours later, the supernatants were collected and used to cultivate the CTLL2 cell line, which is dependent on human IL-2, for 24 hours. The incorporation of ³HT was then measured, as described above. The wells were defined as being positive if the c.p.m. were more than the mean c.p.m. plus 3 standard deviations of the wells with only the receiver's cells. The results were expressed in number of negative wells.

### RT-PCR Amplification and Sequencing Protocols

The total RNA of the clones or cell lines was isolated from 1×10⁶ cells by using the Rnagent kit (Promega, Madison, Wis., United States). The total RNA was converted to single stranded cDNA by using an oligo (dT) primer (Amersham Pharmacia Biotech, Orsay, France) and the reverse transcriptase of the avian myeloblastosis virus, in conformance with the manufacturer's specifications (Promega).

PCR amplification (30 cycles) was conducted using sense 5′ primers specific of the sequence of the 25 V region for TCR-V6 families and a Cβ anti-sense 3′ primer as described in Iwatani et al. (1993) Clin. Exp. Immunol. 93: 430-436. As an internal positive control, primers of region C sense 5′ and anti-sense 3′ were included. The cycles consisted of denaturing steps at 95° C., primer hybridization at 57° C., and extension at 72° C., 1 min each. The PCR was carried out in a Biomed Thermocycler 60 (Biomed Instruments, Fullerton, Calif.) using 2.5 U of Taq DNA polymerase (Cetus, Emeryville, Calif.) in a solution containing 4 pmoles/μl of primers, 0.5 mM of each dNTP; KCl 50 mM; Tris-HCl 10 mM (pH 8.4); MgCl 4 mM and 5 μg of sample. The PCR products were sequenced by Genoscreen (Lille, France). The TCR sequences obtained were analysed using the database IMGT on the Internet (http//:imgt.cines.fr:8104).

### ELISA Assays

1.5×10⁶/ml of T-lymphocyte cell lines or clones were stimulated with 3×10⁶/ml EBV-B LCL irradiated with 50 Gy in X-VIVO-20 medium (Cambrex, Emerainville, France) without serum. The supernatant was eliminated after 24 h. The cytokine concentrations were evaluated by standard ELISA available on the market, in conformance with the manufacturer's instructions. (IL-4, IL-10, IL-2 and IFN-γ (Biosource, Nivells, Belgium), TGF-β (R&D Systems)).

### Characterisation of T-Lymphocyte Epitopes

Three genes encoded by the Y chromosome (DDX3Y, SMCY and RPS4Y) were transfected in male HLA-DR7EBV-B LCL as described in Kinsella and Nolan (1996: Human Gene Therapy 7:1405-1413).

In brief, the two genes encoding DDX3Y and RPS4Y, known to induce the production of HY peptides restricted by the HLA class II molecules, were cloned in the retroviral expression vector pMIGR1 and transfected in the Phoenix-Ampho amphotropic cell line, which expresses the membrane marker IRES-CD8, this having been introduced upstream to the gag-pol construction using lipofectamine. The supernatant containing the viral particles was then extracted from cells that express the CD8 molecule in high concentration, filtered, and concentrated by very rapid centrifugation. It was then used to transduce female HLA-DR7EBV lymphoblastoid cell lines, by infection. More briefly, 8 ml of viral supernatant was mixed with 8 μl of polybrene (Sigma-Aldrich, Gillingham, Dorset, UK) and re-added to 15×10⁶ HLA-DR7EBV-B LCLs. The cells were placed in culture in culture plates with 24 wells (BD Falcon, BD Biosciences, Oxford Science Park, Oxford, UK) in a medium containing RPMI-1640, (Life Technologies, BRL, Paisley, UK) and 10% foetal calf serum (FCS, AutogenBioclear, Caine, UK) for 3 days. The cells were then isolated according to their high rate of expression of the CD8 molecule and the levels of expression of genes encoding DDX3Y and RPS4Y were confirmed by PCR.

HY-specific CD4⁺ T-lymphocytes were stimulated, each with female EBV-B LCL transduced with the HY gene. The proliferation and production of IFN-γ were measured by the incorporation of ³H-[methyl-thymidine] on day 3, and ELISA on day 1. To map the precise T-lymphocyte epitope restricted to the MHC class II, the inventors synthesized several long peptides with a potential bond with DRB1*0701 encoded in the RPS4Y regions which are different from RPS4X, based on several databases for MHC peptide binding, such as SYFPEITHI, Net-MHC, Propred and HotSpot Hunter. These peptides were tested at various concentrations: from 100 μM to 10 nM, using female EBV-B-LCLs that express the HLA-DRB1*0701 molecule in the form of APC. The short peptide in question was identified using a series of peptides of 15 amino acids, also identified from these databases, and which are present in the long peptide which induces a positive response.

### Results

Isolation of CD4/CD8 Double-Positive and CD8⁺ T-Lymphocytes from the Skin of a Patient Suffering from GVHD

One month after the bone marrow transplant between the HLA-identical siblings, where the receiver was a male and the donor a female, the patient/receiver was diagnosed with GVHD of the skin. T-lymphocytes were isolated from the skin biopsy by culture in the presence of 20 U/ml of IL-2. After 14 days in culture, the phenotypic analysis showed two populations of cells, one CD4/CD8 double positive (DP), and the other CD8⁺. The number of CD4/CD8 double positive DP T-lymphocytes was very high, given that they represented almost 55% of the cells. The isolated cells were CD3⁺, but CD56⁻, and 95.9% expressed TCRα/β. When the CD4/CD8 DP T-lymphocytes from skin were sorted with anti-CD4⁺ magnetic beads, stimulated with APCs from the receiver and cultivated without IL-2 for 1 week, the inventors observed that the expression of CD25 was maintained at 84%, CD69 at 58% and CD62L at 61%. Consequently, the inventors were able to isolate two populations of T-lymphocytes from the skin of a patient affected with GVHD; one CD4/CD8 DP, which expresses high levels of CD25 and the other, CD8 single-positive.

Appearance of CD4 Single-Positive Cells after Several Cycles of Stimulation with Receiver's PBMCs

While these two populations were observed at early stages of the culture, after several cycles of stimulation of the sorted CD4/CD8 DP T-lymphocytes with receiver's PBMCs, a new population that expresses only CD4 appeared. This new population was then purified using anti-CD8 magnetic beads. Consequently, after several cycles of stimulation with receiver's PBMCs, three different populations were obtained from the same biopsy of GVHD skin: CD8 single-positive cells, CD4/CD8 DP cells and CD4 single-positive cells.

### VβTCR Spectratyping of the Three Populations

The three sub-populations of sorted cells were then analysed using an immunoscope. The analysis of the GVHD skin T-lymphocytes after 14 days in culture, showed a limited/asymmetrical VOTCR repertoire with oligoclonal expression. After several cycles of stimulation with receiver's PBMCs, the VβTCR repertoire of the three populations was even more asymmetrical. Six fewer VβTCR were represented in the CD8⁺ or CD4⁺ single populations in comparison to those of the CD4/CD8 DP cells. In addition, only two or one new VβTCR appeared in each population, respectively. Interestingly, the inventors noted that by comparing the long-term cultures of CD4/CD8 DP cells with skin T-lymphocytes that were not sorted on day 14, the CD4/CD8 DP population presented a loss of a single VβTCR. This was probably associated with the CD8 profile, given that it was noted in the CD8⁺ single-positive cells, but not in CD4⁺. The appearance of a new VβTCR was also noted in the CD4/CD8 DP cells, and this new VβTCR was also found in the immunoscope profile of the CD4⁺ cells. The observation of such minor modifications in the VβTCR profile at the time of initial screening of CD4/CD8 DP cells suggests that the GVHD skin T-lymphocytes isolated in a 14-day culture were oligoclonal and could be very specific to one or more minor H antigens.

CD4+ T-Lymphocytes Carry Out Proliferative and Cytotoxic Functions after Recognition of an HLA-DR7-Restricted HY Antigen/Epitope.

The inventors analysed the function of CD4⁺ T-lymphocytes by stimulating them with receiver's APCs in the presence of monoclonal antibodies (mAb) directed against HLA molecules. An anti-HLA-DR but not anti-HLA-DQ or HLA-DP mAb significantly inhibited the proliferative response (table 5).

TABLE 5
Functional characterisation of the CD4⁺ T-lymphocyte cell line (I)

| | | + antibody | + antibody | + antibody | + antibody |
|---|---|---|---|---|---|
| ³HT | | α-HLA | α- | α- | α- |
| (c.p.m × 10⁻³) | — | class I | HLA-DR | HLA-DQ | HLA-DP |
| Donor | 615 | ND | ND | ND | ND |
| Receiver | 11188 | 12562 | 886 | 14662 | 9711 |
| HLA-mismatch | 1559 | ND | ND | ND | ND |

ND: not determined

Given that the receiver was homozygous for HLA-DRB1*0701, the inventors then stimulated the CD4⁺ cells with several different male and female APCs that express HLA-DRB1*0701 and noted a clear DR7-restricted anti-HY response, given that the T-lymphocytes responded to all the male HLA-DRB1*0701 cells, but not to the female cells (table 6).

TABLE 6
Functional characterisation of the CD4⁺ T-lymphocyte cell line (II)

| | Receiv- | | | | | | |
|---|---|---|---|---|---|---|---|
| ³HT | er | Male subject | | | Female subject | | |
| (c.p.m × 10⁻³) | APC | APC | | | APC | | |
| HLA-DR7B1*0701 | 12875 | 4552 | 9254 | 9478 | 1170 | 1274 | 838 |
| APC | | | | | | | |

The inventors then tested the cytotoxic activity (CTL) of the CD4⁺ T-lymphocytes, and noted that these cells presented a cytotoxic activity against the receiver's target cells. Once again, it appeared that the limiting element was the HLA-DRB1 molecule, since the cytotoxicity was blocked by an anti-HLA-DR mAb but not an anti-HLA class I mAb, and secondly, cells that express DR81*0701 from a non-relative male donor were lysed by the CD4⁺ T-lymphocyte subset (table 7).

TABLE 7
Functional characterisation of the CD4⁺ T-lymphocyte cell line (III)

| | | | | | | +antibody | +antibody |
|---|---|---|---|---|---|---|---|
| | — | | | | | α-HLA class I | α-HLA-DR |
| Effector/target cell ratio | 10/1 | 3/1 | 1/1 | 0.3/1 | 0.1/1 | 10/1 | 10/1 |

% release of ⁵¹Cr

| | | | | | | | |
|---|---|---|---|---|---|---|---|
| Target donor | 0 | 0 | 0 | 0 | 0 | ND | ND |
| Target receiver | 12 | 12 | 10 | 7 | 4 | 14 | 6 |
| Target PBMC that share | 0 | 0 | 0 | 0 | 0 | ND | ND |
| the HLA class I | | | | | | | |
| Target PBMC that share | 28 | 24 | 17 | 10 | 4 | ND | ND |
| the HLA class II | | | | | | | |

ND: not determined

Differential Profiles of Minor H Antigen Recognition and Expression of Cytokine mRNA by CD4/CD8 DP and CD4⁺ T-Lymphocytes from Skin.

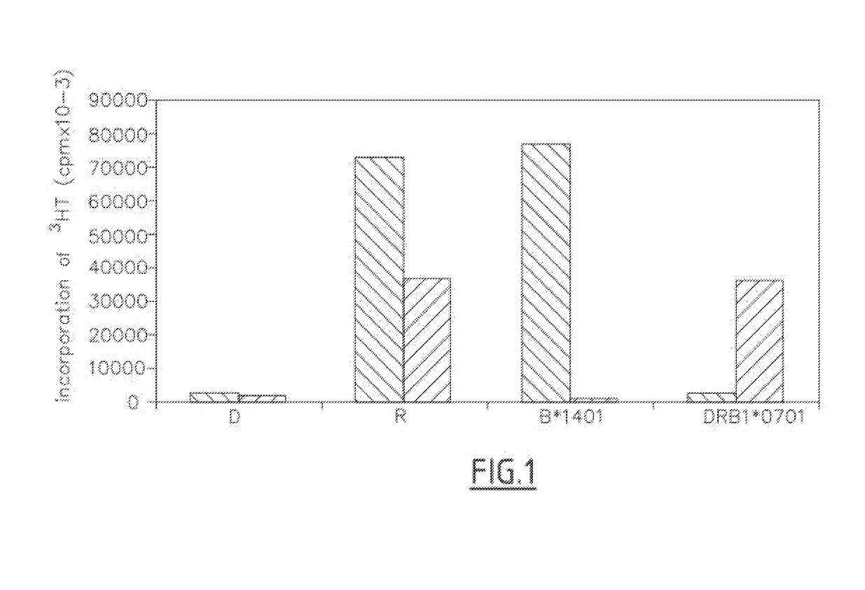

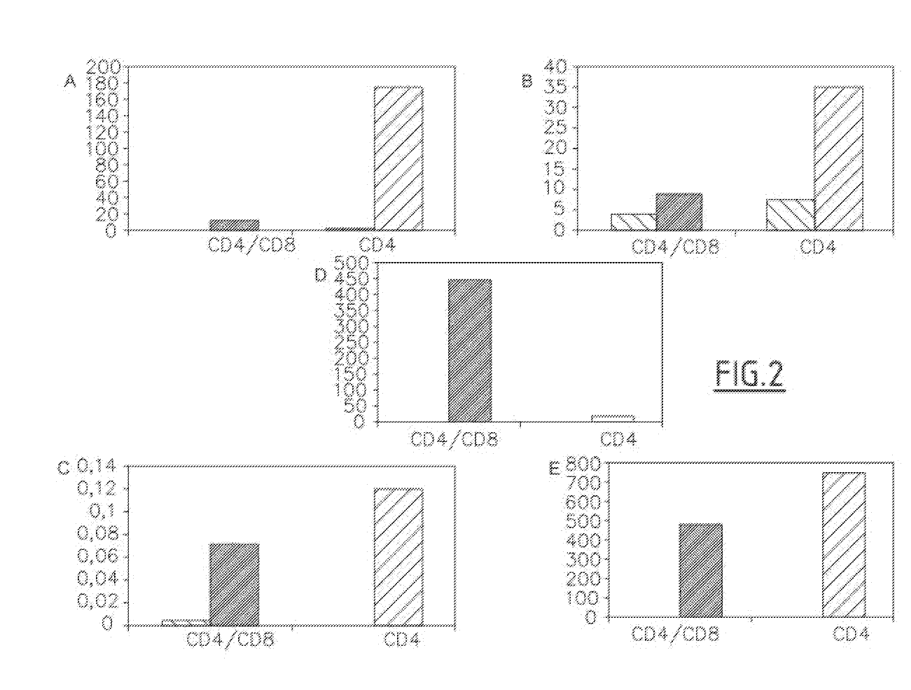

The differential profile for the recognition of minor H antigen by CD4/CD8 DP T-lymphocytes and CD4⁺ T-lymphocytes was confirmed with a proliferative test in which PBMCs that express HLA-B*1401/C*0802 or HLA-DRB1*0701 were used to stimulate the two subsets. As is shown in FIG. 1, the first PBMCs specifically activated the skin CD4/CD8 DP T-lymphocytes, while the second activated the CD4⁺ T-lymphocytes. In addition, after activation by their specific APCs, the mRNA profiles of these two populations were very different. The CD4⁺ T-lymphocytes expressed higher levels of IFNγ, IL-2 and FoxP3, but lower levels of IL-10 mRNA, while the CD4/CD8 DP cells expressed higher levels of MO, but lower levels of FoxP3, IL-2, and IFNγ mRNA (FIG. 2). Although they were expressed in lower levels by the CD4/CD8 DP T-lymphocytes, the IL-4 and TGFβ mRNA were not expressed in a clearly differential manner.

### The HY Epitope Recognized by HLA-DRB1*0701-Restricted CD4⁺ T-Lymphocytes is Encoded by RPS4Y.

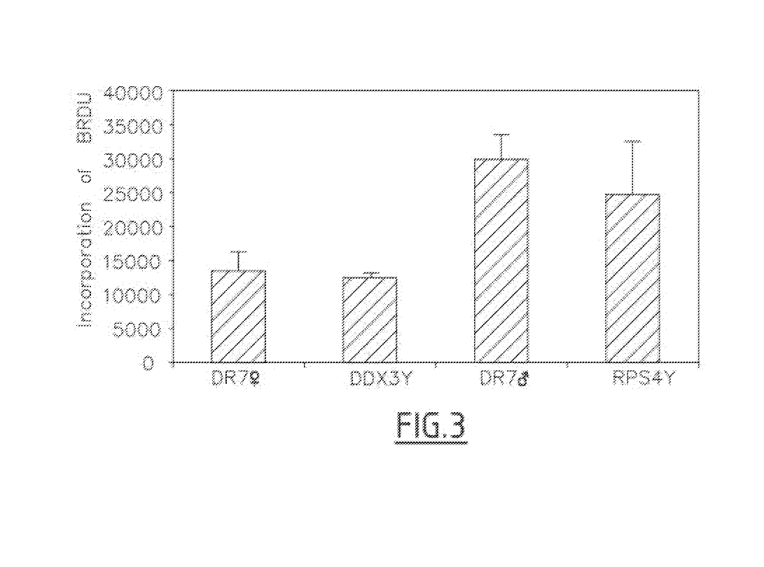

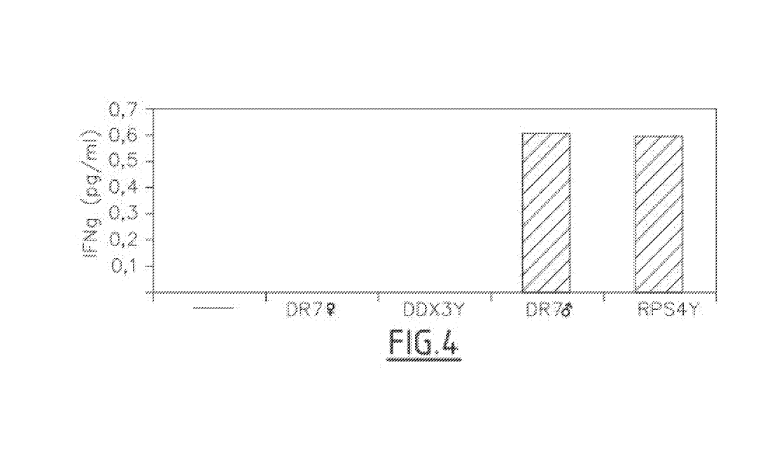

For a more precise definition of which of the HY genes encoded the minor H antigen recognized by the CD4⁺ T-lymphocytes, the inventors transduced female EBV cell lines with the RPS4Y or DDX3Y genes. In parallel, they cloned the CD4⁺ T-lymphocyte cell line and used the production of IFNγ and the proliferation to measure the T-lymphocytes responses. The results showed that RPS4Y, but not DDX3Y was capable of specifically inducing the proliferation of CD4⁺ T-lymphocytes (FIG. 3), as well as the production of IFNγ (FIG. 4).

By using MHC peptide binding databases, the inventors designed peptides approximately 25-40 amino acids long, that covered regions including one or more candidate 15-mer peptide binding motifs to HLA-DRB1*0701, (table 8) and used these to pulse the APCs of female HLA-DR7EBV cells.

TABLE 8
Sequences of the five long peptides used to
stimulate the CD4⁺ T-lymphocyte clone in
the presence of female EBV-BLCL that
express HLA-DRB1*0701

| Long | | |
|---|---|---|
| pep- | | |
| tides | Sequence | Core |
| SK-31 | STGPHKLRECLPLIVF | ECLPLIVFLRNRLKYALTGDE |
| | LRNRLKYALTGDEVK | VKKICMQRFIKIDGKVRVDVT |
| | (SEQ ID NO: 13) | (SEQ ID NO: 17) (Hotspot) |
| | | KLRECLPLIVFLRNRLKYAL |
| | | (SEQ ID NO: 18) (Net MHC) |
| | | LIVFLRNRLKYAL |
| | | (SEQ ID NO: 19) (PROPRED) |
| KM-25 | KICMQRFIKIDGKVRV | KICMQRFIKIDGKVRVDVT |
| | DVTYPAGFM | (SEQ ID NO: 20) (Hotspot) |
| | (SEQ ID NO: 14) | |
| DA-32 | DVISIEKTGEHFRLVY | FRLVYDTKGRFAVHRIT |
| | DTKGRFAVHRITVEEA | (SEQ ID NO: 21) (Hotspot) |
| | (SEQ ID NO: 15) | TKGRFAVHRITV |
| | | (SEQ ID NO: 22) (Hotspot) |
| EV-44 | EEAKYKLCKVRKITVGVK | KYKLCKVRKITVGVKG |
| | GIPHLVTHDARTIRYPDP | (SEQ ID NO: 23) (Hotspot) |
| | (SEQ ID NO: 16) | KYKLCKVRKITVGVKGIPH |
| | | (SEQ ID NO: 24) (Net MHC) |
| | | YKLCKVRKI |
| | | (SEQ ID NO: 25) (PROPRED) |
| | | LVTHDARTI |
| | | (SEQ ID NO: 26) (PROPRED) |
| QR-40 | QIDLGTGKIINFIKFD | GKIINFIKFDTGNLCM |
| | TGNLCMVIGGANLGRV | (SEQ ID NO: 27) (Hotspot) |
| | GVITNRER | TGNVCMVIAGANLGRVG |
| | (SEQ ID NO: 4) | (SEQ ID NO: 28) (Hotspot) |
| | | GGANLGRVGV |
| | | (SEQ ID NO: 29) (Net MHC) |
| | | FIKFDTGNL |
| | | (SEQ ID NO: 30)(PROPRED) |

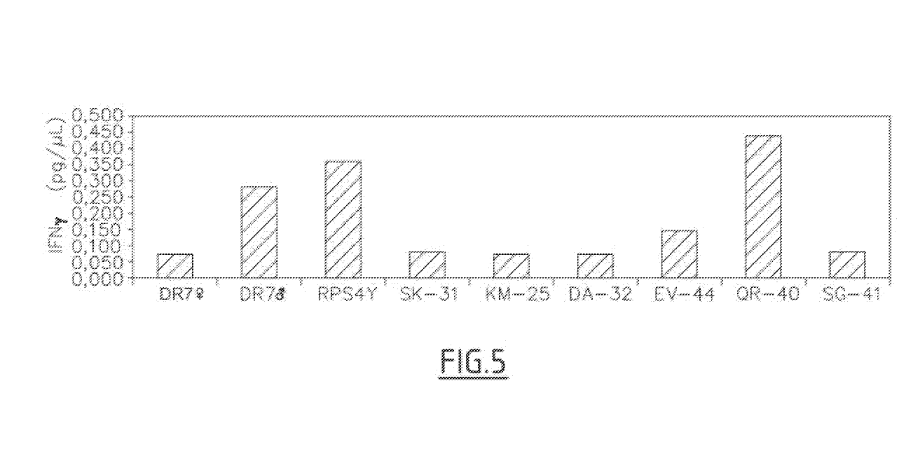

Only one of the 40-mer peptides, QR-40, was capable of stimulating IFNγ production by CD4+ T-lymphocytes (FIG. 5).

The inventors then tested several putative 15-mer peptides in QR-40, and only peptide TL15 stimulated the cells (Table 9).

TABLE 9
Cytokine mRNA expression profile after stimulation of CD4⁺
T-lymphocytes with different short peptides from peptide QR-40.

| | | | | GV17 | | TG17-1 | | TG17-2 | |
|---|---|---|---|---|---|---|---|---|---|
| | | TL15 | | GKIINFIKFDTGNLC | | TGNLCMVIGGANL | | TGNVCMVIAGA | |
| | | TGKIINFIKDTGNL | | MV | | GRVG | | NLGRVG | |
| Peptide | — | (SEQ ID NO: 1) | | (SEQ ID NO: 31) | | (SEQ ID NO: 32) | | (SEQ ID NO: 33) | |
| Concentration | | 10 μM | 1 μM | 10 μM | 1 μM | 10 μM | 1 μM | 10 μM | 1 μM |
| IL2/CD3ε | 0.004 | 0.23 | 0.06 | 0.02 | 0.02 | 0.02 | 0.006 | 0.01 | 0.0047 |
| mRNA ratio | | | | | | | | | |
| IFNγ/CD3ε | 0.15 | 12.63 | 9.91 | 0.82 | 0.12 | 0.11 | 0.11 | 0.5 | 0.009 |
| mRNA ratio | | | | | | | | | |
| FoxP3/CD3ε | 0.009 | 0.44 | 0.42 | 0.27 | 0.12 | 0.32 | 0.12 | 0.3 | 0.23 |
| mRNA ratio | | | | | | | | | |

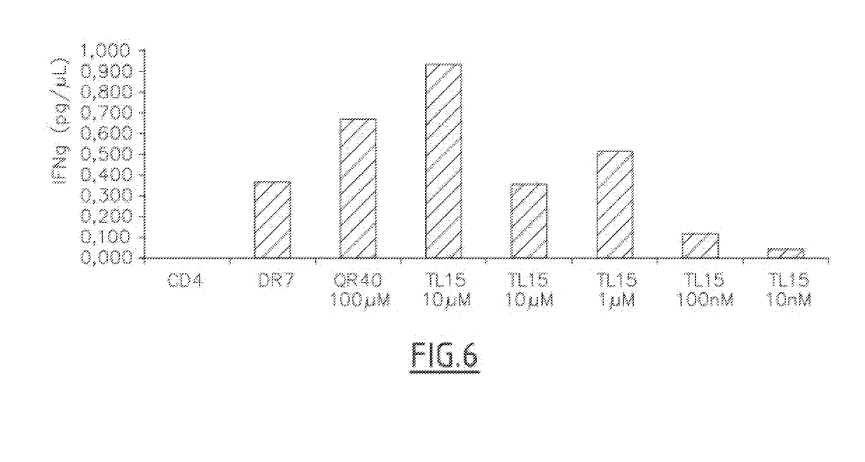

The inventors were therefore able to identify TGKIINFIKFDTGNL (SEQ ID NO: 1) as the HY peptide epitope recognized by the CD4⁺ T-lymphocytes (FIG. 6). The cytokine mRNA profile showed a specific IFNγ induction and a low IL-2 mRNA expression, after activation of the CD4⁺ T-lymphocytes by female HLA-DR7EBV cells pulsed with TL15 (Table 9), while FoxP3 was upregulated in the presence of each peptide, thereby showing its non-specific induction. However, at 1 μM, TL15 induced the highest FoxP3 mRNA response. It is interesting to note that the cloned T-lymphocytes presented the same cytokine profile as the T-lymphocyte cell line.

### RPS4Y-Specific CD4⁺ T-Lymphocytes Express TCRVB19*01

Next, to identify the TCRVβ expressed by the CD4⁺ T-lymphocytes, the inventors sequenced their VβTCR. The results showed that the initial CD4⁺ T-lymphocyte cell line was already clonal, given that only one CDR3 rearrangement was present in the T-lymphocyte cell line and clone. After sequencing, it was noted that the TCRVB/JB/DB regions of the CD4⁺ T-lymphocyte cell line and clone were identical, and homologous with TRBV19*01 and TRBJ2-3*01 from the IMGT database.

### Discussion

In this study, the inventors isolated and characterised a population of high CD4⁺/high CD8⁺ DP T-lymphocytes from the skin of a patient suffering from grade II GVHD, which was rapidly resorbed. It is interesting to note that the donor/receiver pair was HLA-identical, but not gender-matched in the direction that promotes activation of T-lymphocytes from the HY-specific female donor by the male receiver's APCs. This CD4/CD8 DP population was co-isolated with a single population of CD8⁺ T-lymphocytes.

The CD4/CD8 DP T-lymphocyte subset was noted only in the patient's skin and not in peripheral blood. This subset specifically proliferated in response to antigens expressed by the receiver's cells.

Interestingly, after repeated cycles of stimulation with receiver APCs, the inventors also isolated a subset of CD4⁺ T-lymphocytes from the same cultures. These cells were capable of producing IL-2 and IFNγ and of carrying out cytotoxic functions (FIG. 2 and tables 5 to 7). An additional characterisation of CD4 single-positive T-lymphocytes demonstrated that the minor H antigen that they recognize was different from the one recognized by the CD4/CD8 DP T-lymphocytes. Indeed, the inventors formally excluded the possible recognition of an HY antigen by the CD4/CD8 DP T-lymphocytes, while the CD4⁺ T-lymphocytes were clearly HY-specific (table 6). In addition, the CD4/CD8 DP T-lymphocytes were HLA class I-restricted, while the CD4⁺ T-lymphocytes were HLA class II-restricted, which makes it unlikely that the CD4⁺ T-lymphocytes were directly derived from the CD4/CD8 DP T-lymphocytes but rather that they were amplified during the repeated in vitro culture. Their cytokine profile was also different; the CD4/CD8 double T-lymphocytes predominantly expressed transcripts for IL-10 and TGFβ, while the CD4⁺ T-lymphocytes preferentially induced IFNγ and FoxP3 mRNA after activation (FIGS. 1 and 2). Finally, the inventors observed that the VβTCR profiles of these two populations were distinct, given that some VβTCR were lost in the CD4 single-positive population, in relation to the CD4/CD8 DP T-lymphocytes. It is interesting to note that only TCRVB7 appeared in the CD4⁺ population.

Given that it was noted that the CD4⁺ T-lymphocytes were HY-specific and HLA-DR7-restricted, the inventors then attempted to identify the gene and the peptide that encode this HY epitope, by using a candidate gene approach similar to the one previously used to identify the murine MHC class II-restricted HY epitopes. With the use of a retrovirus, they transduced female DR7-EBV cells with the candidate DDX3Y and RPS4Y genes. By using the isolated CD4 single-positive T-lymphocyte clone or cell line, they identified RPS4Y as the gene that expressed the DR7-restricted HY epitope. To identify the peptide epitope itself, they first used long peptides of 25 to 40 amino acids, designed to incorporate candidate DR7 binding peptides that were identified in several databases such as SYFPEITHI, Net-MHC, Propred and HotSpot Hunter. This enabled them to define a peptide, QR40, as one that expresses the epitope (table 8), and led them to identify the specific 15-mer peptide, TGKIINFIKFDTGNL (SEQ ID NO: 1) as the HY peptide epitope recognized by the CD4⁺ T-lymphocytes. However, by comparing the response of the CD4⁺ T-lymphocyte clone to the long and short peptides, they observed that the shortest peptide, which did not require treatment, induced higher levels of response (FIG. 4). This epitope is clearly new. Although it was already shown that RPS4Y encodes an HLA-class II-restricted HY epitope, and induces a helper and cytotoxic activity, in this study the epitope was HLA-DRB3-restricted.

In conclusion, this example describes a new HLA class II-restricted HY minor H antigen, its amino acid sequence, its HLA-restriction and the CDR3 region of the VβTCR by which it is recognized. Given that the HLA class II minor H antigens have been described as a putative target of leukaemia cells, in vivo, due to their predominant expression by haematopoietic stem cells, these observations should contribute to specifically targeting male HLA-DR7 leukaemia cells, in cell therapy programmes.

1. An isolated polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3).

2. The polypeptide according to claim 1, the said polypeptide consisting of the sequence TGKIINFIKFDTGNL (SEQ ID NO: 1).

3. A nucleic acid comprising or consisting of a sequence that encodes a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3).

4. A vector comprising a nucleic acid according to claim 3, in which the said nucleic acid is operatively bound to one or more elements enabling the expression of the polypeptide.

5. A method for producing a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
said method comprising the steps that consist of:
a) chemically or enzymatically synthesizing the said polypeptide or inducing the expression of the said polypeptide by a cell comprising a nucleic acid according to claim 3 or a vector comprising said nucleic acid, in which the said nucleic acid is operatively bound to one or more elements enabling the expression of the polypeptide; and
b) retrieving the said polypeptide obtained in step a).

6. A method of in vitro preparation of T-lymphocytes capable of specifically recognizing an epitope from (i) a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or from (ii) a polypeptide comprising the complete sequence of the protein encoded by the RPS4Y gene
and presented by the HLA-DR7 molecule expressed on the surface of antigen-presenting cells, comprising the steps of:
T-lymphocyte co-culture with antigen-presenting cells that express on their surface the HLA-DR7 molecule to which is attached an epitope from (i) the polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or from (ii) a polypeptide comprising the complete sequence of the protein encoded by the RPS4Y gene; and
retrieval of the T-lymphocytes from the said co-culture.

7. An isolated T-lymphocyte that specifically recognizes an epitope from (i) a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or from (ii) a polypeptide comprising the complete sequence of the protein that is encoded by the RPS4Y gene
and presented by the HLA-DR7 molecule expressed on the surface of antigen-presenting cells, wherein the said T-lymphocyte is likely to be obtained by the method of preparation according to claim 6.

8. A pharmaceutical composition comprising (i) a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or a T-lymphocyte as defined in claim 7 and (ii) a pharmaceutically acceptable vehicle.

9. A medicament comprising (i) a polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or (ii) a T-lymphocyte as defined in claim 7.

10. A method for treating a cancer of immune cells, in which a therapeutically effective quantity of (i) an isolated polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or of (ii) an isolated T-lymphocyte as defined in claim 7, is administered to a patient in need thereof.

11. A method for treating a cancer of immune cells in a subject who expresses the HLA-DR7 molecule, in which a therapeutically effective quantity of (i) an isolated polypeptide comprising:
a) the TGKIINFIKFDTGNL sequence (SEQ ID NO: 1), or
b) a peptide fragment of at least 9 consecutive amino acids of the sequence SEQ ID NO: 1 or
c) a variant of the sequence SEQ ID NO: 1 or of a fragment according to b), differing from the sequence SEQ ID NO: 1 or from the fragment according to b) by conservative substitution of one, two or three amino acids, or
d) a peptidomimetic of the sequence SEQ ID NO: 1 or of the fragment according to b) or of the variant according to c), said peptidomimetic being obtained by structural modification using unnatural amino acids,
the said peptide fragment, the said peptidomimetic and the said variant having the capacity to activate T-lymphocytes when it is presented by the HLA-DR7 molecule;
the said polypeptide comprising neither the complete sequence of the protein encoded by the RPS4Y gene, nor the TIRYPDPVI sequence (SEQ ID NO: 2), nor the VIKVNDTVQI sequence (SEQ ID NO: 3),
or of (ii) an isolated T-lymphocyte as defined in claim 7, or of (iii) a polypeptide comprising the complete sequence of the protein encoded by the RPS4Y gene, is administered in said subject in need thereof.


SEQUENCE LISTING (33 sequences)

SEQ ID NO: 1 - protein, 15 aa, Artificial Sequence
  peptide TL15
TGKIINFIKFDTGNL

SEQ ID NO: 2 - protein, 9 aa, Artificial Sequence
  peptide described in Ivanov et al, excluded from the invention
TIRYPDPVI

SEQ ID NO: 3 - protein, 10 aa, Artificial Sequence
  peptide described in Spierings et al, excluded from the invention
VIKVNDTVQI

SEQ ID NO: 4 - protein, 40 aa, Artificial Sequence
  Peptide QR-40
QIDLGTGKIINFIKFDTGNLCMVIGGANLGRVGVITNRER

SEQ ID NO: 5 - protein, 263 aa, Homo sapiens
MARGPKKHLKRVAAPKHWMLDKLTGVFAPRPSTGPHKLRECLPLIVFLRNRLKYALTGDEVKKICMQRFIKIDGKVRVDVTYPAGFMDVISIEKTGEHFRLVYDTKGRFAVHRITVEEAKYKLCKVRKITVGVKGIPHLVTHDARTIRYPDPVIKVNDTVQIDLGTGKIINFIKFDTGNLCMVIGGANLGRVGVITNRERHPGSFDVVHVKDANGNSFATRLSNIFVIGNGNKPWISLPRGKGIRLTVAEERDKRLATKQSSG

SEQ ID NO: 6 - protein, 9 aa, Artificial Sequence
  First fragment of 9 consecutive amino acids of SEQ ID NO 1
TGKIINFIK

SEQ ID NO: 7 - protein, 9 aa, Artificial Sequence
  Second fragment of 9 consecutive amino acids of SEQ ID NO 1
GKIINFIKF

SEQ ID NO: 8 - protein, 9 aa, Artificial Sequence
  Third fragment of 9 consecutive amino acids of SEQ ID NO 1
KIINFIKFD

SEQ ID NO: 9 - protein, 9 aa, Artificial Sequence
  Fourth fragment of 9 consecutive amino acids of SEQ ID NO 1
IINFIKFDT

SEQ ID NO: 10 - protein, 9 aa, Artificial Sequence
  Fifth fragment of 9 consecutive amino acids of SEQ ID NO 1
INFIKFDTG

SEQ ID NO: 11 - protein, 9 aa, Artificial Sequence
  Sixth fragment of 9 consecutive amino acids of SEQ ID NO 1
NFIKFDTGN

SEQ ID NO: 12 - protein, 9 aa, Artificial Sequence
  Seventh fragment of 9 consecutive amino acids of SEQ ID NO 1
FIKFDTGNL

SEQ ID NO: 13 - protein, 31 aa, Artificial Sequence
  Peptide SK-31
STGPHKLRECLPLIVFLRNRLKYALTGDEVK

SEQ ID NO: 14 - protein, 25 aa, Artificial Sequence
  Peptide KM-25
KICMQRFIKIDGKVRVDVTYPAGFM

SEQ ID NO: 15 - protein, 32 aa, Artificial Sequence
  Peptide DA-32
DVISIEKTGEHFRLVYDTKGRFAVHRITVEEA

SEQ ID NO: 16 - protein, 36 aa, Artificial Sequence
  Peptide EV-44
EEAKYKLCKVRKITVGVKGIPHLVTHDARTIRYPDP

SEQ ID NO: 17 - protein, 42 aa, Artificial Sequence
  Core motif from the Hotspot database, source of peptide SK-31
ECLPLIVFLRNRLKYALTGDEVKKICMQRFIKIDGKVRVDVT

SEQ ID NO: 18 - protein, 20 aa, Artificial Sequence
  Core motif from the Net MHC database, source of peptide SK-31
KLRECLPLIVFLRNRLKYAL

SEQ ID NO: 19 - protein, 13 aa, Artificial Sequence
  Core motif from the Propred database, source of peptide SK-31
LIVFLRNRLKYAL

SEQ ID NO: 20 - protein, 19 aa, Artificial Sequence
  Core motif from the Hotspot database, source of peptide KM-25
KICMQRFIKIDGKVRVDVT

SEQ ID NO: 21 - protein, 17 aa, Artificial Sequence
  Core motif from the Hotspot database, source of peptide DA-32
FRLVYDTKGRFAVHRIT

SEQ ID NO: 22 - protein, 12 aa, Artificial Sequence
  Second core motif from the Hotspot database, source of peptide DA-32
TKGRFAVHRITV

SEQ ID NO: 23 - protein, 16 aa, Artificial Sequence
  Core motif from the Hotspot database, source of peptide EV-44
KYKLCKVRKITVGVKG

SEQ ID NO: 24 - protein, 19 aa, Artificial Sequence
  Core motif from the Net MHC database, source of peptide EV-44
KYKLCKVRKITVGVKGIPH

SEQ ID NO: 25 - protein, 9 aa, Artificial Sequence
  Core motif from the Propred database, source of peptide EV-44
YKLCKVRKI

SEQ ID NO: 26 - protein, 9 aa, Artificial Sequence
  Second core motif from the Propred database, source of peptide EV-44
LVTHDARTI

SEQ ID NO: 27 - protein, 16 aa, Artificial Sequence
  Core motif from the Hotspot database, source of peptide QR-40
GKIINFIKFDTGNLCM

SEQ ID NO: 28 - protein, 17 aa, Artificial Sequence
  Second core motif from the Hotspot database, source of peptide QR-40
TGNVCMVIAGANLGRVG

SEQ ID NO: 29 - protein, 10 aa, Artificial Sequence
  Core motif from the Net MHC database, source of peptide QR-40
GGANLGRVGV

SEQ ID NO: 30 - protein, 9 aa, Artificial Sequence
  Core motif from the Propred database, source of peptide QR-40
FIKFDTGNL

SEQ ID NO: 31 - protein, 17 aa, Artificial Sequence
  Peptide GV17
GKIINFIKFDTGNLCMV

SEQ ID NO: 32 - protein, 17 aa, Artificial Sequence
  Peptide TG17-1
TGNLCMVIGGANLGRVG

SEQ ID NO: 33 - protein, 17 aa, Artificial Sequence
  Peptide TG17-2
TGNVCMVIAGANLGRVG